(19) United States
(12) Patent Application Publication
(10) Pub. No.: US 2026/0226910 A1
(43) Pub. Date: Aug. 6, 2026

(54) IMPELLER OF A PUMP, AND PUMP

(71) Applicant: Rheinisch-Westfalische Technische Hochschule Aachen, Abgektirzt RWTH Aachen Korperschaft des dffe, Aachen (DE)

(72) Inventors: Sebastian Victor JANSEN, Aachen (DE); Michelle SCHUELLER, Aachen (DE); Ulrich STEINSEIFER, Aachen (DE); Jan SYGOR, Aachen (DE); Thomas SCHMITZ-RODE, Aachen (DE)

(21) Appl. No.: 19/149,746

(22) PCT Filed: Jan. 10, 2024
(86) PCT No.: PCT/EP2024/050446

§ 371 Date: Jul. 21, 2025

(30) Foreign Application Priority Data
Jan. 25, 2023 (DE) 10 2023 101 857.4

(51) Int. Cl. F04D 29/24; F04D 13/02

(52) U.S. Cl. CPC F04D 29/247; F04D 13/024; F04D 29/242

(57) ABSTRACT

The invention relates to an impeller of a pump, in particular a blood pump, comprising a plurality of vanes, which have magnetically acting coupling elements disposed around an axis of rotation, wherein the impeller can in particular be caused to rotate in a contactless manner as a result of magnetic interaction between the coupling elements and a rotating magnetic field, wherein each of the vanes has at least two action regions, which are spaced apart from and flexibly connected to one another and on which forces can be exerted by means of a magnetic field, by which the relative position of the action regions with respect to one another can be changed. The invention also relates to a pump comprising a pump housing including an impeller of the described type comprising vanes.

The invention relates to an impeller of a pump, preferably of a radial pump, in particular of a blood pump, comprising multiple vanes, which has magnetically acting coupling elements disposed around an axis of rotation. In this way, the impeller can preferably be caused to rotate in a contactless manner as a result of magnetic interaction between the coupling elements and a rotating magnetic field. Such a rotating magnetic field can be caused to rotate, for example, by a drive of a pump, which is designed to create a rotated magnetic field.

The invention also relates to a pump, preferably a radial pump, in particular a blood pump, comprising a pump housing including an impeller comprising vanes, which comprises magnetically acting coupling elements, and comprising a magnetically acting drive for creating at least one rotating magnetic field, by way of which the impeller can be caused to rotate in a contactless manner. This is achieved by the magnetic interaction between the rotating magnetic field of the drive and the magnetically acting coupling elements of the impeller. Such a magnetic field preferably acts through a wall of the pump housing on the impeller disposed in the pump housing.

The impeller in the pump is preferably driven by the rotating magnetic field and is preferably also mounted in the pump, in particular is hydrodynamically mounted in the fluid flowing around the impeller, preferably in a liquid, in particular blood. The mounting is thus preferably achieved by the cooperation of magnetically and hydrodynamically generated forces. The impeller, however, can preferably also be point-mounted or spherically mounted in the pump, for example on/at a ball disposed in the pump, and in particular in the pump housing.

In the description of the present invention, a magnetically acting coupling element shall be understood to mean an element on which a force can be exerted by a magnetic field. The coupling element itself can be magnetic or at least magnetizable.

A coupling element shall preferably be understood to mean a magnetically acting element, which, for the purpose of driving the impeller in the pump housing of the pump or for other purposes according to the invention, cooperates with a rotating magnetic field, in particular of a pump drive.

Such impellers and pumps comprising such impellers are generally known in the prior art, for example from the publication WO 2014/000753 A1 by the same applicant.

In medical technology, radial pumps are used as blood pumps to replace or support the heart, both in implantable form and in extracorporeal applications. For hemocompatibility reasons, no dynamic seals can be used in blood pumps, which is why the impeller in the pump housing is typically driven in a contactless manner by magnetic forces. Even though blood pumps (as well as all radial pumps) are designed for a fixed operating point, these are frequently used over a wide operating point range. Operating the pumps outside the operating point, however, can cause clinical complications, such as hemolysis and thrombus formation in the pump.

The invention also preferably relates to the use of the impeller and of the pump in the clinical field of application of blood pumps, without being limited thereto.

Against this background, it is an object of the invention to provide an impeller and a pump comprising such an impeller which can be adapted to different operating conditions.

According to the invention, this is achieved in that each of the vanes has at least two action regions, which are spaced apart from and flexibly connected to one another and on which forces can be exerted by means of a magnetic field, by which the relative position of the action regions with respect to one another can be changed.

Such an impeller has the advantage that the vanes thereof are not rigid, as is typically the case, so that the flexibility of the vanes opens up the possibility of the vanes becoming deformed.

As a result of the at least two provided action regions, which are spaced apart from and flexibly connected to one another, a movement of at least one action region relative to at least one another action region can be created by the action of a magnetically created force.

Such a relative movement thus impacts the shape of the vanes and/or the arrangement thereof relative to the remaining components of the impeller. Overall, the impeller geometry can thus be changed and, for example, as a result of such a change, a geometric adaptation of the impeller, and in particular of the vanes thereof, to the present operating point of an operated pump can be carried out.

It is preferably provided that an action of a force changing the impeller geometry acts simultaneously, and in particular acts identically, on all vanes of the impeller. This ensures that all vanes of the impeller are influenced, and in particular are deformed, or that the arrangement thereof relative to other impeller regions is changed in the same manner by the magnetic field.

Preferably, a magnetic field that is utilized to change the impeller geometry as well as the magnetic field driving the impeller are rotated about an axis of rotation about which the impeller rotates. It is furthermore preferred that the magnetic field that is utilized to change the impeller geometry also acts in a driving manner on the impeller. Still more preferably, at least two rotating magnetic fields cooperate in a pump, so as to drive the impeller of the pump and influence the impeller geometry thereof, in particular by changing the cooperation between the at least two magnetic fields, preferably by changing the superimposition of the cooperating magnetic fields.

An action region of a vane shall be understood to mean a region of a vane on which a magnetically created force acts, or to which the force is transferred, when the impeller is located in a magnetic field, which is in particular artificially created, preferably in a drive of a pump in which the impeller is inserted. An action region is therefore itself magnetic or at least magnetizable or mechanically connected to a magnetic or magnetizable region/part of the impeller.

It is preferably provided that a respective action region comprises therein at least one magnetically acting coupling element or is connected to at least one magnetically acting coupling element.

A coupling element comprised by the vane is preferably integrated into the material that forms the vane. A connection between an action region and at least one coupling element can be carried out via other regions of the impeller, which in particular are not part of the vane, for example via regions on/to which a respective vane is attached, such as, for example, by way of the impeller bodies described below.

A coupling element, as mentioned at the outset, shall be understood to mean a magnetically acting element to which at least one rotating magnetic field created by a pump drive couples, which is to say, exerts driving forces, and/or which is utilized for the magnetically created change in geometry.

The invention thus preferably achieves that the use of coupling elements in the impeller can effectuate both the driving thereof and the change in the geometry thereof. Preferably, this also opens up the option of effectuating a change in geometry indirectly by driving a pump, and in particular during the operation of the pump.

Particularly preferably, it is provided that the impeller comprises at least two groups of magnetically acting coupling elements, and each of the vanes has at least two spaced-apart action regions, to each of which at least one coupling element of another group is assigned, in particular by a connection between the action region and the at least one assigned coupling element or by integration of the at least one assigned coupling element into the action region, wherein the impeller geometry can be changed by a relative movement, in particular about the axis of rotation, between the at least two groups of coupling elements.

So as to make such a relative movement possible, the different groups of coupling elements in the impeller are preferably not connected among one another, or are only flexibly connected to one another, in particular indirectly via flexible regions of the vanes.

It is preferably provided in the design that the at least two groups of coupling elements and/or the at least two action regions of a particular vane are spaced apart in a radial direction. The radial direction shall be understood with respect to the axis of rotation about which the impeller is to rotate or rotates during operation in a pump.

Within a particular group, the coupling elements of this group are preferably all disposed around the axis of rotation, in particular in each case at the same angular spacing between the coupling elements.

The coupling elements belonging to a group are preferably all coupling elements of the impeller which are located on the same radius or radial interval. On the different vanes, the coupling elements of a group are always assigned to an action region that is the same. Action regions that are the same are preferably all those action regions of all vanes that are located on the same radius or radial interval. As a result, action regions at different radial positions are assigned to different groups of coupling elements. The groups of coupling elements thus influence all vanes in the same manner.

The invention can preferably provide that the coupling elements of one of the at least two groups are at least predominantly provided to exert the driving force on the impeller by means of a rotating magnetic field acting on these coupling elements. It may be provided that the coupling elements of another group also act in a driving manner, in particular, however, are predominantly provided to exert a force on the assigned action regions of the vanes so that one action region of a vane is displaced in relation to another action region.

A coupling element of a group is preferably considered to be assigned to an action region when a connection, in particular a rigid connection, exists between the coupling element and the action region. The connection shall at least be understood to be rigid when no relative movement occurs between the at least one coupling element and the assigned action region as a result of magnetic forces that act during an operation of a pump.

At least one coupling element of a group, or all coupling elements of a group, can be disposed in the impeller, spaced apart in the axial direction from the vanes of the impeller, and in particular can be located in an impeller body axially beneath a connecting region in which the vanes are attached to the impeller body.

Preferably, the number of coupling elements present in each group of coupling elements matches the number of vanes disposed at the impeller.

Coupling elements of a group and assigned action regions of the vanes can, for example, be disposed so that, in each case, one coupling element and one action region are aligned in the axial direction, in particular are aligned among one another in a direction parallel to the axis of rotation. It may also be provided that the coupling elements of a group are located at a different radial position than the assigned action regions. This allows a magnetic action direction to be generated, which deviates from a direction parallel to the axis of rotation, for example 45 degrees in relation thereto. Coupling elements of a group and assigned action regions of the vanes can also be disposed so as to be spaced apart in the axial direction of the axis of rotation, and so as to be offset from one another about the axis of rotation in the circumferential direction. In such an arrangement, the coupling elements of a group are located between two assigned action regions, viewed in the circumferential direction about the axis of rotation.

Different action regions of a vane are connected to coupling elements of different groups, so that a force can be generated between spaced-apart action regions by different forces that act on the coupling elements of different groups.

The described relative movement can thus be caused by different magnetically created forces which act on the coupling elements of different groups, in particular during pump operation. Such forces can be generated by magnetic fields that act differently on the coupling elements of different groups. This can also take place in a stable manner during a rotation of the impeller during operation of a pump when the magnetic fields which act on the coupling elements themselves also rotate, and in particular drive the impeller so as to rotate.

Changing the impeller geometry preferably means changing a vane parameter, preferably changing the spatial position of the vanes in the impeller, in particular the position relative to the axis of rotation, and/or changing the inflow and/or outflow angles, and/or changing the wrap angle, and/or changing the vane shape (for example the curvature), and/or changing the direction of the extension of the vane, in particular in every alternative in relation to an impeller region that co-rotates with the vanes in the impeller, such as the impeller described hereafter. In particular, the inflow angle can also be referred to as inlet angle and the outflow angle can be referred to as outlet angle of the vane.

In particular, the inflow angle/inlet angle is measured between the tangents to the vane at the radially inner end thereof and/or the tangent to the circle on which the radially inner end of the vane is located. In particular, the outflow angle/outlet angle is measured between the tangents to the vane at the radially outer end thereof and/or the tangent to the circle on which the radially outer end of the vane is located. In particular, the wrap angle is the angular spacing on the circle on which the radially outer end of the vane is located, between the radially outer end of the vane and the radial projection of the radially inner end of the vane. Each of the described circles extends around the axis of rotation of the impeller comprising the vanes.

The vanes are preferably made of a flexible, preferably elastic, material, which in particular has greater flexibility than other regions/elements of the impeller, in particular regions/elements that connect, preferably rigidly, the coupling elements of a group among one another, such as of the impeller body described below. In this way, an action region can be moved relative to another by externally applying magnetically acting forces to the impeller on each vane. For example, the vanes can be made of an elastomer, for example silicone, or of sheet metal, for example sheet titanium. For example, an impeller body described below can be made of a plastic material or of another, preferably non-magnetic/non-magnetizable material.

The object is thus also achieved by a pump in which the impeller is designed according to one embodiment of the description of the present invention, and at least two magnetic fields, which can be rotated/rotate about the axis of rotation of the impeller and are phase-shiftable relative to one another, can be created by way of the drive, wherein each or the rotatable/rotating magnetic fields cooperates with another group of coupling elements of the impeller.

The number of rotatable magnetic fields that can be created by way of the drive preferably corresponds to the number of groups of coupling elements in the impeller, and in particular also to the number of action regions on each vane.

Preferably, the at least two rotating magnetic fields have the same frequency during the rotation so that the phase shift is stable during the rotation, and the geometry of the impeller is likewise stable, in particular in terms of the vanes thereof.

The phase shift can preferably be in the range of 0 to 10 degrees, more preferably 0 to 20 degrees, still more preferably 0 to 30 degrees, and more preferably 0 to 40 degrees.

The coupling elements of all groups in the impeller are preferably designed as permanent magnets, but can alternatively also be designed as coils. It may also be provided in the impeller that at least one group of permanent magnets and at least one group of coils are installed.

In terms of the pump, it is preferably provided that the drive comprises multiple drive units, in particular drive rings, which are concentric around the axis of rotation and preferably disposed inside one another and which in the angular positions around the axis of rotation are phase-shiftable with respect to one another, wherein each drive unit comprises a group of coupling elements, in particular permanent magnets and/or coils that magnetically cooperate with an axially opposing group of coupling elements of the impeller.

Each drive unit can be rotated, for example by means of a motor, about the axis of rotation. Each drive unit can comprise a dedicated motor, wherein the phase shift can be generated by differing activation of the motors. All drive units can also be driven by the same motor, wherein the phase shift between two drive units can be generated by an adjustable gear connection acting between these units.

It may also be provided for a drive to comprise two drive units having a fixedly defined phase shift, which cannot be changed during operation.

Such a drive can then only move the impeller in a fixedly defined geometry which does not change as long as the drive is mounted.

By switching the drive to a drive having a different phase shift, a different, fixedly defined geometry can be generated. In this case, a different drive having an accordingly defined phase shift can be provided for every desired change in geometry.

It may also be provided for the drive to comprise at least one arrangement of stationary coils, by way of which, as a result of electrical activation, at least two rotating magnetic fields which are phase-shiftable relative to one another can be created, wherein each of the created magnetic fields cooperates with another group of coupling elements of the impeller.

Preferably, a dedicated arrangement of stationary coils can be provided for each of the rotatable magnetic fields. In particular, a drive comprising stationary coils does not comprise any mechanically moving parts.

Likewise, it may be provided for the drive to comprise at least one arrangement of stationary coils for creating a rotating magnetic field and at least one drive unit, which comprises coupling elements and can be rotated about the axis of rotation, for creating at least one further rotating magnetic field, wherein each of the rotating magnetic fields cooperates with another group of coupling elements in the impeller.

According to a preferred exemplary design implementation of the impeller, the impeller comprises an impeller body, in which one of the at least two groups of coupling elements is disposed, wherein all radially inner action regions, and in particular the radially inner ends of the vanes, are attached to this impeller body, in particular to the upper side thereof, and the radially outer action regions, and in particular radially outer ends of the vanes, form free action regions that protrude in the radial direction to the outside beyond the impeller body, wherein at least one coupling element of at least one other group is disposed in the radially outer action region of a particular vane.

As an alternative, the impeller body can form a ring, to which the radially outer action regions, in particular the radially outer ends of the vanes, are attached, in particular to the surface thereof, and the radially inner action regions, in particular radially inner ends of the vanes, form free action regions that protrude in the radial direction to the inside beyond the impeller body, wherein at least one coupling element of another group is disposed in the radially inner action region of a particular vane.

The upper side of the impeller body is preferably the side of the impeller body which faces away from the drive or the driving magnetic field.

A free action region shall preferably be understood to mean that this region is freely movable in the circumferential direction about the axis of rotation, in particular at least within the scope of the flexibility thereof. This region is preferably not connected in the circumferential direction to other free action regions of other vanes.

In this embodiment, the coupling elements of at least one group are disposed directly within the vanes, namely in the respective free action region of the vanes, preferably in each case exactly one coupling element per free action region.

Between the action region of a vane which is attached to the coupling element and a free action region, in particular at the radial end of a vane, a vane can have at least one further (free) action region, which is not connected in the circumferential direction to a neighboring vane, and in particular is integrated into the (exactly) one coupling element.

In these embodiments having free action regions, the invention can furthermore provide that the free action regions protrude, in the axial direction, beyond the surface of the impeller body at which the vane is attached to the other action region. The vane can thus have a larger axial length at the free action region than at the action region of the vane which is attached to the impeller body.

It can also be provided here that, proceeding from the action region that is attached to the impeller body, the thickness of a vane, viewed in the circumferential direction around the axis of rotation, increases toward the free action region. This preferably creates space in the free action region so as to receive the coupling element therein.

In another preferred embodiment, it is provided that the impeller comprises an impeller body that is divided into at least two body rings, which are concentric with respect to the axis of rotation and located inside one another and which can be rotated relative to one another about the axis of rotation.

The number of body rings preferably corresponds to the number of groups of coupling elements, wherein the coupling elements of a different group are disposed in each body ring, and the action regions connected to the coupling elements of a particular group are attached to the body ring which comprises the coupling elements of this particular group.

The coupling elements of the group contained in a particular body ring are rigidly connected among one another by that ring. A flexible connection can exist between such body rings, preferably via flexible regions of the vanes.

The body ring located radially the furthest to the inside is also referred to as a ring, even if it does not have the cut-out surrounding the axis of rotation. Usually, at least in the case of blood pumps, such a central cut-out is present to allow blood to flow through, for example so as to achieve hydrodynamic mounting of the impeller in the blood or avoid stagnation of blood between the impeller and the housing wall or to cool a mount.

Such an impeller body of the above-described types can also be referred to as an impeller cup.

In all possible embodiments, it is preferably provided that an action region, and in particular each, action region connected to the impeller body, in particular to a body ring of the impeller body, is either non-rotatably connected to the impeller body, in particular to the body ring, or is connected rotatably, in particular freely rotatably or rotatably against a restoring force, to the impeller body, in particular to the body ring.

In the case of a non-rotatable connection of the action region, in particular of an action region located radially the furthest to the inside and/or outside, preferably no influence is exerted by a relative movement on the outflow angle and/or inflow angle of the vane. In contrast, the extension/shape of the vane can change between spaced-apart action regions.

The inflow angle and/or outflow angle are changed in the case of the rotatable attachment, in particular of an action region that is located radially the furthest to the inside and/or outside.

A restoring force can, for example, be generated by the rotatable connection between the action region and the impeller body, in particular the body ring, being formed by a twistable rod, which is anchored in the impeller body on the one hand and in the action region on the other hand so that the twisting between the action region and the impeller body is carried out by the torsion of the rod. Such a rod is preferably oriented axially, which is to say parallel with respect to the axis of rotation.

In a further preferred refinement, it is provided that the impeller body, and in particular each body ring of the impeller body, comprises a lower body part and an upper body part, between which the vanes are disposed, wherein the attached action regions are preferably each non-rotatably or rotatably attached in the upper and lower body parts.

A further preferred refinement can provide that two radially neighboring body rings are braced on one another in the axial direction, for which purpose stepped surfaces are preferably formed in the mutually radially opposing surfaces of the neighboring body rings, which are preferably located opposite one another around a shared plane that is perpendicular to the axis of rotation or inclined with respect to the axis of rotation. Such stepped surfaces are preferably axially aligned, which is to say have a normal vector parallel to the axis of rotation or a normal vector that is inclined with respect to the axis of rotation, in particular perpendicular to the aforementioned plane.

The invention can additionally preferably provide that the vane has no contact with the body rings between two radially spaced-apart action regions which are connected to body rings, and in particular are non-rotatably connected. In this way, the vane can freely flexibly deform between the action regions.

More preferably, the coupling elements of all groups are disposed in a shared plane that is perpendicular to the axis of rotation or around a shared plane that is perpendicular to the axis of rotation.

Exemplary embodiments of the invention will be described based on the figures.

The following is a brief description of the drawings:

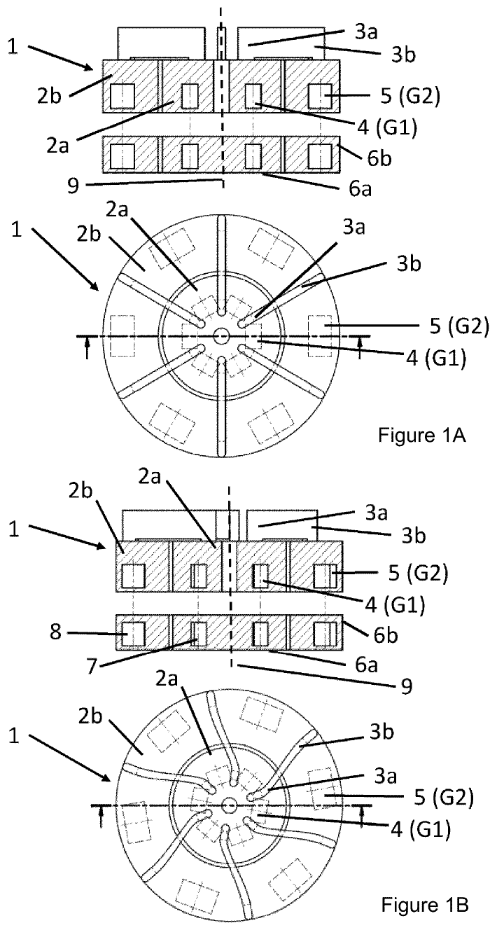

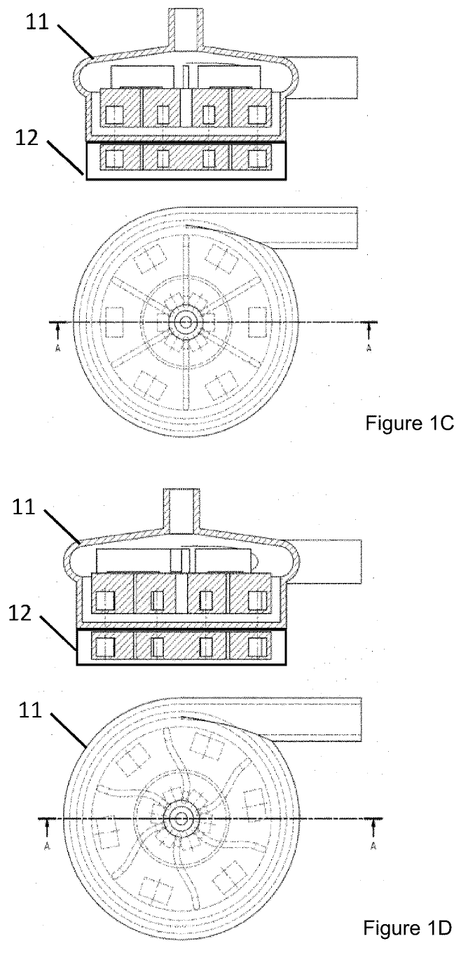

FIGS. 1A and 1B show a first embodiment of an impeller of the invention;

FIGS. 1C and 1D show the arrangement of the impeller and drive according to FIGS. 1A and 1B within a pump;

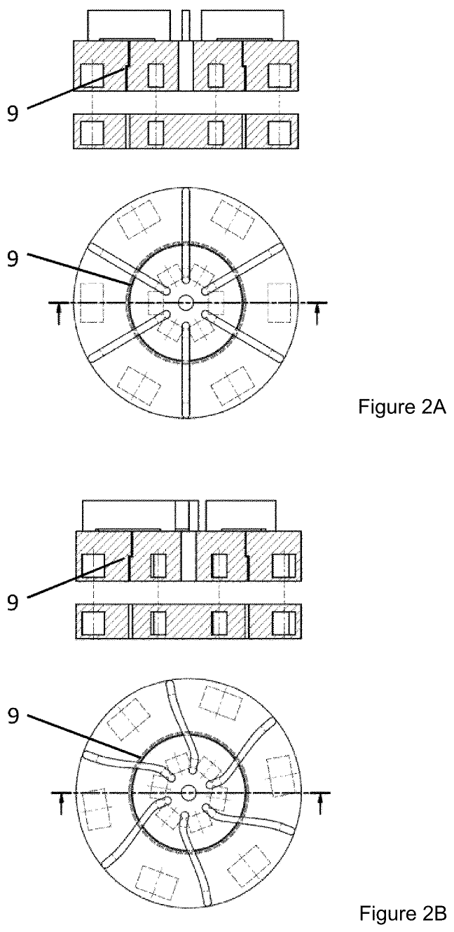

FIGS. 2A, 2B show a modification in which the body rings brace one another in the axial direction;

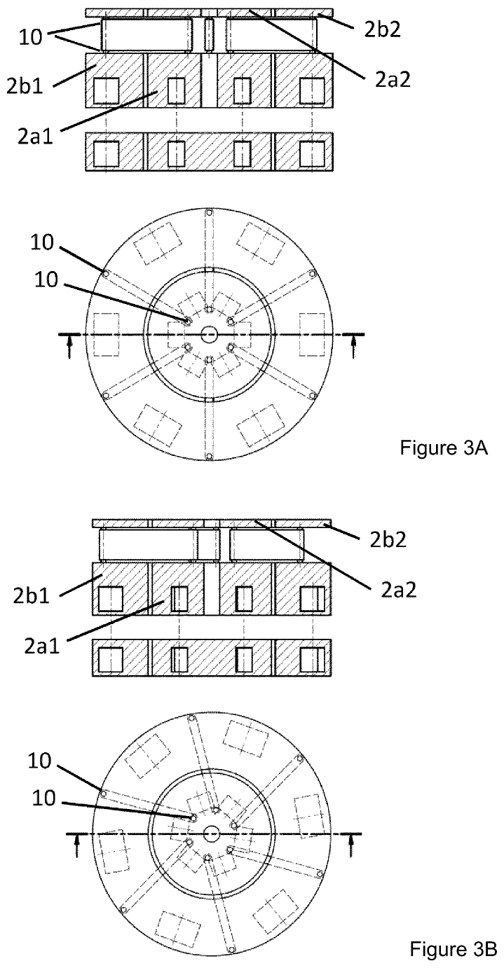

FIGS. 3a, 3b show a further modification;

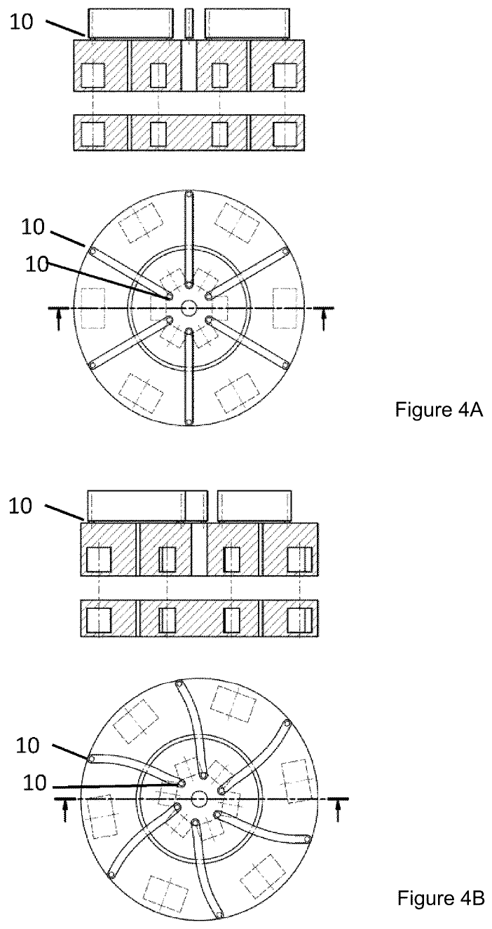

FIGS. 4A, 4B show an embodiment according to FIGS. 2A, 2B in which the only change is that the non-rotatable attachment of the action region to the body rings is replaced with rotatable attachments by means of pivot bearings;

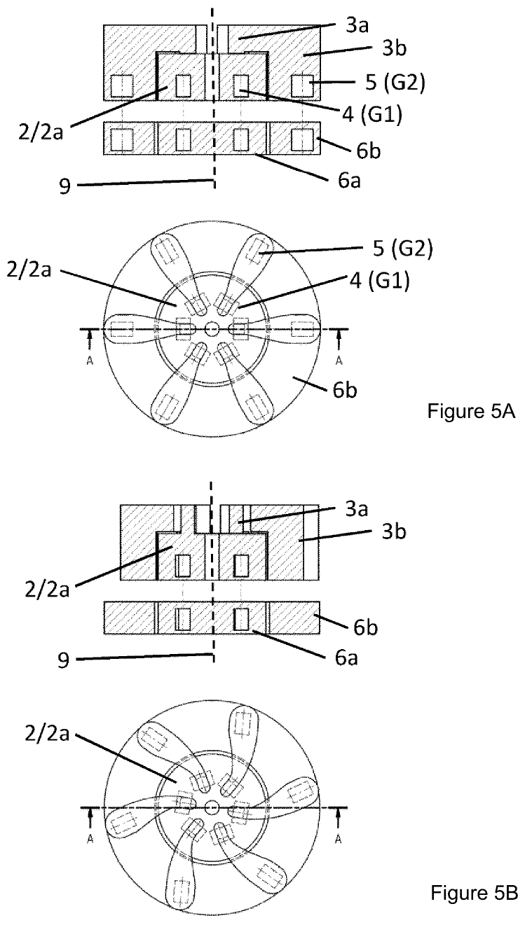

FIGS. 5a, 5b show a modification for all other embodiments; and

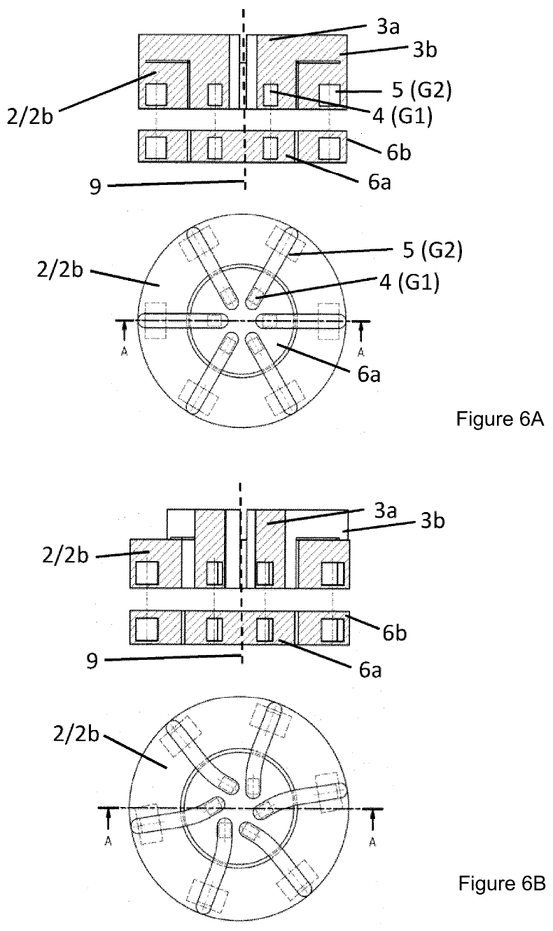

FIGS. 6A, 6B show a modification compared to the embodiment of FIGS. 5A, 5B.

FIGS. 1A and 1B show a first embodiment of an impeller of the invention, located axially opposite two drive rings 6a, 6b of a pump drive of a pump, which is not shown otherwise, in two different states. The entire pump, in contrast, is shown in FIGS. 1C and 1D. Here as well as in the other figures, it is to be assumed that the impeller is disposed in a pump housing of the pump and separated by a housing wall from the drive rings 6a, 6b of the pump drive, through which the magnetic fields created by the drive rings 6a, 6b act.

In the embodiment shown in FIGS. 1A and 1B, the impeller 1 comprises a two-piece impeller body 2, which includes a radially inner body ring 2a and a radially outer body ring 2b. A gap is provided between the body rings 2a, 2b. In the radial direction, the body rings 2a and 2b are only connected via the vanes 3, which are made of a flexible material, for example an elastomer. Here and in all other embodiments, flexible shall be understood to mean that the vanes 3 are able to deform or become displaced in relation to the impeller body 2 as a result of magnetically acting forces.

Each of the vanes 3 here has two action regions 3a and 3b, wherein the action regions 3a, 3b are formed by the radial inner and outer ends of the vanes 3. The vanes 3 are attached, and in the present exemplary embodiment are non-rotatably attached, with the action regions 3a, 3b to the surface of the body rings 2a, 2b. Between the action regions 3a, 3b, the vanes 3 have no contact with the surface of the body rings 2a, 2b.

A first group (G1) of permanent magnets, serving as coupling elements 4, is disposed in the inner body ring 2a, and a second group (G2) of permanent magnets, serving as coupling elements 5, is disposed in the outer body ring 2b. Due to the mechanical connection, the action regions 3a are assigned to the first group G1 of coupling elements 4, and the action regions 3b are assigned to the second group G2 of coupling elements 5. The coupling elements 4, 5 are located axially spaced-apart beneath the action regions 3a, 3b and offset therefrom in the circumferential direction. In an axial top view onto the impeller, the coupling elements 4, 5 are thus located between the action regions 3a, 3b of vanes 3 adjoining one another in the circumferential direction.

No rigid connection is present either between the action regions 3a, 3b or between the coupling elements 4, 5 of the two groups, but rather only the flexible connection is present via the flexible regions of the vane 3, which extend in the radial direction between the action regions 3a, 3b.

The drive, which here is located axially beneath the impeller, comprises two drive rings 6a, 6b, which are preferably disposed so as to be axially aligned with the body rings 2a, 2b and in turn comprise coupling elements 7 and 8. The inner drive ring 6a is also referred to as a ring, even though in this embodiment there is no central opening.

Two, preferably independent, rotating magnetic fields can be created by way of the coupling elements 7 of the inner drive ring 6a and the coupling elements 8 of the outer drive ring 6b by rotation of the drive rings 6a, 6b about the axis of rotation 9, so that the inner body ring 2a of the impeller can be driven by way of the inner drive ring 6a and the coupling elements 7 thereof as a result of the magnetic interaction with the coupling elements 4 of the first group G1, and the outer body ring 2b of the impeller can be driven by way of the outer drive ring 6b and the coupling elements 8 thereof as a result of the magnetic interaction with the coupling elements 5 of the second group G2. The impeller as a whole is thus driven by both drive rings 6a, 6b and the two magnetic fields created thereby.

A phase shift of the rotational angle position of the two drive rings 6a, 6b with respect to one another creates a phase shift between the rotating magnetic fields, which is transferred to the body rings 2a and 2b, so that, depending on the set phase shift, different forces can be exerted on the action regions 3a, 3b of the vanes 3, which are connected via the body rings 2a, 2b to the two groups G1, G2 of the coupling elements 4 and 5.

FIG. 1A shows a first possible phase position, and FIG. 1B shows a second possible phase position, which is to say a shift of the phases between the situations shown in FIGS. 1A and 1B.

It is apparent that, according to FIG. 1A, the vanes are all exactly aligned in a radial rectilinear manner as a result of the forces that act on the action regions 3a, 3b of the vanes 3.

In FIG. 1B, in contrast, different forces are acting on the action regions 3a, 3b, whereby these, compared to the situation shown in FIG. 1A, change the position thereof relative to the body rings 2a, 2b, and the vanes 3 change the shape thereof. This way, a change in the impeller geometry is achieved, in which the inflow and outflow angles of the vanes remain the same due to the non-rotatable attachment of the action regions 3a, 3b to the body rings 2a, 2b, and the wrap angles of the vanes 3 change.

This change in the geometry of the impeller can be effectuated simply by a change in the phase position of the drive rings 6a, 6b with respect to one another.

FIGS. 1C and 1D show the arrangement of the impeller and drive according to FIGS. 1A and 1B within a pump. The impeller 1 is surrounded by a pump housing 11. The drive rings 6a and 6 can be surrounded by a drive housing/motor housing 12. The created magnetic fields act through the housing wall of the pump housing 11 and/or drive housing/motor housing 12. For the sake of clarity, the remaining reference numerals which are identical to FIGS. 1A and 1B are not shown in FIGS. 1C and 1D.

Compared to FIGS. 1A, 1B, FIGS. 2A, 2B show a modification in which the body rings 2a, 2b brace one another in the axial direction. For this purpose, a respective step 9 is formed in the radially mutually opposing circumferential surfaces of the body rings 2a, 2b. The axially oriented surfaces of the step 9 contact one another.

All remaining features of FIGS. 1A, 1B and 2A, 2B coincide. The embodiment according to FIGS. 2A, 2B can likewise be disposed in a pump housing and/or drive housing/motor housing, similarly to FIGS. 1C and 1D.

FIG. 3 show a further modification in which, compared to FIGS. 1A, 1B and 2A, 2B, the body rings 2a, 2b each comprise an axially lower body ring (2a1, 2b1) and upper body ring (2a2, 2b2) between which the vanes are disposed.

In contrast to FIGS. 1A, 1B and 2A, 2B, the vanes are furthermore attached with the action regions 3a, 3b thereof to the body rings 2a and 2b so as to be rotatable about pivot bearings 10, but could also be non-rotatably attached. All remaining features coincide with those in FIGS. 1 and 2. Pivot bearings 10 can be free rotatable or, for example, be implemented by a twistable rod to which the action regions are attached.

All remaining features of FIGS. 1A, 1B, 2A, 2B and 3A, 3B coincide. The embodiment according to FIGS. 3A, 3B can likewise be disposed in a pump housing and/or drive housing/motor housing, similarly to FIGS. 1C and 1D.

FIGS. 4A, 4B show an embodiment according to FIGS. 2A, 2B in which the only change is that the non-rotatable attachment of the action regions 3a, 3b to the body rings 2a, 2b is replaced with rotatable attachments by means of pivot bearings 10.

All remaining features of FIGS. 2A, 2B and 4A, 4B coincide. The embodiment according to FIGS. 4A, 4B can likewise be disposed in a pump housing and/or drive housing/motor housing, similarly to FIGS. 1C and 1D.

Compared to FIGS. 1A, 1B and 2A, 2B, FIGS. 3A, 3B and 4A, 4B show that the inflow angle and the outflow angle of the vanes 3, at the radial ends thereof, change with the phase shift between the drive rings 6a, 6b and the body rings 2a, 2b, as a result of the respective rotatable attachment between the action regions 3a, 3b and the body rings 2a, 2b.

FIGS. 5A, 5B show a modification for all other embodiments, according to which the impeller body 2 only comprises a radially inner body ring 2a in which the coupling elements 4 of the first group G1 are disposed. The vanes 3 are attached with the radially inner action regions 3a thereof to this impeller body 2.

The radially outer ends of the vanes 3 protrude beyond the body ring 2a, forming there respective free action regions 3b that, viewed in the circumferential direction around the axis of rotation 9, are not connected among one another. The coupling elements 4 of the first group G1 are disposed in the impeller body 2 here, as well as in the other embodiments, however are not offset from the action regions, but are axially spaced apart therefrom and aligned therewith, that is, directly beneath the action regions. The coupling elements 5 of the second group G2, in contrast, are directly integrated into the action region 3b of the vanes 3, and in particular the coupling elements 5 of the second group are thus not rigidly connected among one another. The coupling elements of the two groups G1 and G2 are located at the same axial position.

The drive rings 6a, 6b are designed as in the other figures.

Similarly to the other embodiments, the free action regions 3b can be moved relative to the action regions 3a by a change in the phase position (rotational angle position) of the drive rings 6a, 6b with respect to one another, whereby the wrap angle of the vanes 3 changes significantly.

So as to be able to receive the coupling elements 5, the radially outer ends of the vanes 3 are designed to be thicker than the radially inner ends.

The embodiment according to FIGS. 5A, 5B can likewise be disposed in a pump housing and/or drive housing/motor housing, similarly to FIGS. 1C and 1D.

FIGS. 6A, 6B show a modification compared to the embodiment of FIGS. 5A, 5B, according to which the impeller body 2 only comprises a radially outer body ring 2b in which the coupling elements 5 of the second group G2 are disposed. The vanes 3 are attached with the radially outer action regions 3b thereof to this impeller body 2, and in this case are non-rotatably attached.

The radially inner ends of the vanes 3 protrude beyond the body ring 2b, forming there respective free action regions 3a that, viewed in the circumferential direction around the axis of rotation 9, are not connected among one another. The coupling elements 5 of the second group G2 are disposed in the impeller body 2 here, as well as in the other embodiments, however are not offset from the action regions, but are axially spaced apart therefrom and aligned therewith, that is, directly beneath the action regions. The coupling elements 4 of the first group G1, in contrast, are directly integrated into the action region 3a of the vanes 3, and in particular the coupling elements 4 of the first group are thus not rigidly connected among one another. The coupling elements of the two groups G1 and G2 here are located at the same axial position, as is the case in all shown embodiments.

The drive rings 6a, 6b are designed as in the other figures.

Similarly to the other embodiments, the free action regions 3a can be moved relative to the action regions 3b by a change in the phase position (rotational angle position) of the drive rings 6a, 6b with respect to one another, whereby the wrap angle of the vanes 3 changes significantly.

The radially inner ends of the vanes 3 are designed to be sufficiently thick so as to be able to receive the coupling elements 4, however in particular not thicker than the radially outer ends.

The embodiment according to FIGS. 6A, 6B can likewise be disposed in a pump housing and/or drive housing/motor housing, similarly to FIGS. 1C and 1D.

1. An impeller of a pump comprising a plurality of vanes, magnetically acting coupling elements disposed around an axis of rotation and configured to cause the impeller to rotate in a contactless manner as a result of magnetic interaction between the coupling elements and a rotating magnetic field, wherein each of the vanes has at least two action regions which are spaced apart from and flexibly connected to one another and on which forces can be exerted by means of a magnetic field, by which a relative position of the action regions with respect to one another can be varied.

2. The impeller according to claim 1, wherein each of the action regions comprises at least one magnetically acting coupling element therein or is connected to at least one magnetically acting coupling element.

3. The impeller according to claim 1, wherein
a. the impeller comprises at least two groups of the magnetically acting coupling elements that are spaced apart in a radial direction, and
b. each of the vanes has at least two of the action regions that are spaced apart in the radial direction, to each of which at least one of the coupling elements of another of the groups is assigned by a connection between the action region and the at least one assigned coupling element or by integration of the at least one assigned coupling element into the action region,
c. with the impeller geometry being changeable by a relative movement about the axis of rotation between the at least two groups of coupling elements.

4. The impeller according to claim 1, wherein the impeller comprises an impeller body in which one of the groups of coupling elements is disposed, wherein
a. radially inner ends of the vanes are attached to a surface of the impeller body radially outer ends of the vanes form the free action regions which protrude beyond the impeller body to the outside in the radial direction, at least one of the coupling elements of at least one other of the groups being disposed in the radially outer action region of a particular one of the vanes, or
b. the impeller body forms a ring to a surface of which the radially outer ends of the vanes are attached, and radially inner ends of the vanes form the free action regions that protrude in the radial direction to the inside beyond the impeller body, at least one coupling element of another of the groups being disposed in the radially inner action region of a particular one of the vanes.

5. The impeller according to claim 4, wherein the free action regions protrude in the axial direction beyond the surface of the impeller body to which the particular one of the vanes is attached with an other of the action regions.

6. The impeller according to claim 5, wherein thickness of each of the vanes viewed in the circumferential direction around the axis of rotation increases from the action region that is attached to the impeller toward the free action region.

7. The impeller according to claim 3, wherein the impeller body is divided into at least two body rings which are concentric with respect to the axis of rotation and located inside one another and which can be rotated relative to one another about the axis of rotation the number of body rings corresponding to the number of groups of coupling elements, the coupling elements of a different one of the groups being disposed in each of the body rings and the action regions connected to the coupling elements of a particular one of the groups being attached to the body ring which comprises the coupling elements of that particular one of the groups.

8. The impeller according to claim 7, wherein each of the action regions connected to one of the body rings of the impeller body
a. is connected non-rotatably to the body ring, or
b. is connected freely rotatably or rotatably against a restoring force to the body ring.

9. The impeller according to claim 1, wherein each of the body rings of the impeller body comprises a lower and an upper body part between which the vanes are disposed, the attached action regions being each non-rotatably or rotatably attached in the upper and lower body parts.

10. The impeller according to claim 9, wherein two radially neighboring ones of the body rings are braced on one another in the axial direction, for which purpose stepped surfaces are formed in mutually radially opposing surfaces of the neighboring body rings, which surfaces are located opposite one another around a shared plane that is perpendicular to the axis of rotation.

11. The impeller according to claim 10, wherein each of the vanes has no contact with the body rings between two radially spaced-apart action regions which are non-rotatably connected to body rings.

12. The impeller according to claim 11, wherein the coupling elements of all the groups are disposed in a shared plane that is perpendicular to the axis of rotation or around a shared plane that is perpendicular to the axis of rotation.

13. The impeller according to claim 3, the coupling elements of all of the groups are permanent magnets.

14. A pump comprising a pump housing including an impeller comprising vanes, which impeller has magnetically acting coupling elements and comprises a drive unit for creating at least one rotating magnetic field by way of which the impeller can be caused to rotate in a contactless manner, wherein the impeller comprises the impeller according to claim 3, and at least two magnetic fields that are rotatable/rotate about the axis of rotation of the impeller and that are phase-shiftable relative to one another are created by way of the drive, each of the rotatable magnetic fields cooperating with a different group of coupling elements of the impeller.

15. The pump according to claim 14, wherein the drive comprises a plurality of the drive units, the drive units each comprising a respective drive ring, the drive rings being concentric about the axis of rotation and disposed inside one another and which in the angular positions are phase-shiftable with respect to one another about the axis of rotation each of the drive units comprising a group of permanent magnets or coils that magnetically cooperate with an axially opposing one of the groups of coupling elements of the impeller.

16. The pump according to claim 14, wherein the drive comprises at least one arrangement of stationary coils by way of which, as a result of electrical activation, at least two rotating magnetic fields which are phase-shiftable relative to one another can be created, each of the created magnetic fields cooperating with a different one of the groups of coupling elements of the impeller.

17. The pump according to claim 14, wherein the drive comprises at least one arrangement of stationary coils for creating a rotating magnetic field and at least one drive unit which comprises coupling elements and can be rotated about the axis of rotation for creating at least one further rotating magnetic field, each of the rotating magnetic fields cooperating with another of the groups of coupling elements in the impeller.
